(19) United States
(12) Patent Application Publication
(10) Pub. No.: US 2023/0293741 A1
(43) Pub. Date: Sep. 21, 2023

(54) ULTRAVIOLET LIGHT IRRADIATION SYSTEM AND DECONTAMINATION METHOD

(71) Applicant: NIPPON TELEGRAPH AND TELEPHONE CORPORATION, Tokyo (JP)

(72) Inventors: Takashi MATSUI, Musashino-shi, Tokyo (JP); Kazuhide NAKAJIMA, Musashino-shi, Tokyo (JP); Nobutomo HANZAWA, Musashino-shi, Tokyo (JP); Yuto SAGAE, Musashino-shi, Tokyo (JP); Chisato FUKAI, Musashino-shi, Tokyo (JP); Ayako IWAKI, Musashino-shi, Tokyo (JP); Tomohiro TANIGUCHI, Musashino-shi, Tokyo (JP); Kazutaka HARA, Musashino-shi, Tokyo (JP); Atsuko KAWAKITA, Musashino-shi, Tokyo (JP)

(73) Assignee: NIPPON TELEGRAPH AND TELEPHONE CORPORATION, Tokyo (JP)

(21) Appl. No.: 18/018,035

(22) PCT Filed: Oct. 21, 2020
(86) PCT No.: PCT/JP2020/039544

§ 371 Date: Jan. 25, 2023

(30) Foreign Application Priority Data
Jul. 30, 2020 (WO) PCT/JP2020/029269

(51) Int. Cl. A61L 2/10; G02B 6/10; A61L 2/26; A61L 2/24

(52) U.S. Cl. CPC A61L 2/10; A61L 2/24; A61L 2/26; G02B 6/102; A61L 2202/11; A61L 2202/14

(57) ABSTRACT

An object of the present invention is to provide an ultraviolet light irradiation system and a decontamination method that are economical and easy to operate, and that can perform decontamination without any input from a user. In the present invention, an optical fiber or an optical waveguide that radiates ultraviolet light in a lateral direction, the optical fiber or the optical waveguide is built in a sheet shape, and irradiates a surface with the ultraviolet light. Specifically, a material in which a material having a high scattering coefficient is added to the optical fiber is used, a grating is formed in the optical fiber, the optical fiber is given a minute bending with a minute ruggedness, and an arbitrary bending is given on the optical fiber, or the like, thereby achieving side radiation. By such a feature, it has the effect that ultraviolet light decontamination can be performed at all times or at necessary timing for an object touched by an unspecified number of people.

### TECHNICAL FIELD

The present disclosure relates to an ultraviolet light irradiation system and a decontamination method for performing sterilization and virus inactivation using ultraviolet light.

### BACKGROUND ART

For the purpose of preventing infectious diseases, there is an increasing demand for systems with ultraviolet light for sterilization and virus inactivation using ultraviolet light. In the present embodiment, “decontamination” is assumed to include sterilization and virus inactivation.

There are three major categories of products in decontamination systems.

### Mobile Sterilization Robot

A mobile sterilization robot is an autonomous mobile robot for radiating ultraviolet light, (for example, refer to Non Patent Literature 1). The mobile sterilization robot radiates ultraviolet light while moving in a room in a building such as a hospital room, thereby automatically performing decontamination in a wide range without manual operation.

### Stationary Air Purifier

A stationary air purifier is a device that is installed on a ceiling or in a predetermined place in a room and decontaminates while circulating the air in the room (for example, refer to Non Patent Literature 2). Since a stationary air purifier does not emit ultraviolet light to the outside and has no influence on the human body, it is capable of highly safe decontamination.

### Portable Sterilization Device

A portable sterilization device is a portable type device mounted with an ultraviolet light source such as a fluorescent lamp, a mercury lamp, or an LED (for example, refer to Non Patent Literature 3) A user brings a portable sterilization device to an area that the user wants to decontaminate, and irradiates the area with ultraviolet light. In this way, a portable sterilization device can be used in various locations.

### CITATION LIST

### Non Patent Literature

Non Patent Literature 1: Website of Kantum Ushikata Co., LTD. (https://www.kantum.co.jp/product/sakkin_robot/sakkinn_robot/U VD_robot), search on Jul. 3, 2020
Non Patent Literature 2: Website of IWASAKI ELECTRIC CO., LTD. (https://www.iwasaki.co.jp/optics/sterilization/air/air03.html ), search on Jul. 3, 2020
Non Patent Literature 3: Website of Funakoshi Co., Ltd. (https://www.funakoshi.co.jp/contents/68182), search on Jul. 3, 2020

### SUMMARY OF INVENTION

### Technical Problem

The purpose of the techniques disclosed so far is to specify an object and a range, or to perform decontamination limited thereto. Therefore, these techniques require that decontamination be performed every time a person uses a train, a machine, or the like used by an unspecified number of people, and their operations are complicated.

The prior art further has the following difficulties.

Since the mobile sterilization robot radiates high-power ultraviolet light, the device is large and expensive. Therefore, it is difficult to economically realize the mobile type sterilization robot.

Since the stationary air purifier uses a method of sterilizing the circulated indoor air, there is a problem that it is difficult to decontaminate clothes and the like and to immediately decontaminate bacteria and viruses emitted from carriers.

The portable sterilization device has a problem that the irradiated ultraviolet rays are relatively weak and it is difficult to use it to decontaminate in a short time. Even if a mercury lamp or a fluorescent lamp with high output is used, these lamps are generally large-sized and short-lived, and since light is diffused in proportion to the square of the distance to reduce the power, it is difficult to apply these lamps to a portable sterilization device.

Although implementation of sterilization systems and methods for solving these problems are expected, no specific means has come to light. Therefore, in order to solve the above problems, an object of the present invention is to provide an ultraviolet light irradiation system and a decontamination method that are economical and easy to operate, and that can perform decontamination without any input from a user.

### Solution to Problem

In order to achieve the above object, in the ultraviolet light irradiation system according to the present invention, an optical fiber or an optical waveguide radiates ultraviolet light in a lateral direction, the optical fiber or the optical waveguide is built in a sheet shape, and irradiates a surface with the ultraviolet light

Specifically, the ultraviolet light irradiation system according to the present invention includes
an ultraviolet light source unit for outputting ultraviolet light, and
a sheet on which an optical waveguide that laterally emits the ultraviolet light in a longitudinal direction is disposed.

In addition, a decontamination method according to the present invention includes
attaching a sheet on which an optical waveguide that laterally emits light in a longitudinal direction is disposed to a desired object, and
inputting ultraviolet light into the optical waveguide of the sheet.

An object can be decontaminated by attaching the sheet to the object that is touched by an unspecified number of people and allowing ultraviolet light to leak from the sheet at all times or at a required timing. In this way, the ultraviolet light irradiation system can perform decontamination easily without any input from a user. Accordingly, the present invention can provide an ultraviolet light irradiation system and a decontamination method that are economical and easy to operate, and can perform decontamination without any input from a user.

The ultraviolet light irradiation system according to the present invention further includes a plurality of the sheets, and

a branch switch unit configured to branch the ultraviolet light output from the ultraviolet light source unit and supply the branched ultraviolet light to the optical waveguide of each sheet, or sequentially supply the ultraviolet light output from the ultraviolet light source unit to the optical waveguide of each sheet.

Since one ultraviolet light source can decontaminate multiple locations, it is economical.

The ultraviolet light irradiation system according to the present invention further includes

a sensor for detecting approach of a human body; and an irradiation control unit configured to control output or non-output of the ultraviolet light from the ultraviolet light source unit based on a signal of the sensor.

Safety can be improved and the service life of the equipment can be prolonged.

The ultraviolet light irradiation system according to the present invention further includes:

a visible light source that outputs visible light in synchronization with the output or the non-output of the ultraviolet light of the ultraviolet light source unit, and an optical multiplexing unit that multiplexes the visible light output from the visible light source with the ultraviolet light output from the ultraviolet light source unit.

Thus, it can be clearly indicated that it is in operation for improved safety.

For example, the optical waveguide may be provided with predetermined bending or micro-bending. In addition, the optical waveguide may have a plurality of bubbles or gratings in a waveguide region of the ultraviolet light.

In addition, the optical waveguide includes any one of a solid core optical fiber, a hole assist optical fiber, a hole structure optical fiber, a hollow core optical fiber, a coupled core type optical fiber, a solid core type multi-core optical fiber, a hole assist type multi-core optical fiber, a hole structure type multi-core optical fiber, a hollow core type multi-core optical fiber, and a coupled core type multi-core optical fiber.

The inventions can be combined with each other where possible.

### Advantageous Effects of Invention

The present invention can provide an ultraviolet light irradiation system and a decontamination method that are economical and easy to operate, and that can perform decontamination without any input from a user.

### BRIEF DESCRIPTION OF DRAWINGS

FIG. 1 is a diagram illustrating an ultraviolet light irradiation system according to the present invention.

FIG. 2 is a diagram illustrating the ultraviolet light irradiation system according to the present invention.

FIG. 3 is a diagram illustrating the ultraviolet light irradiation system according to the present invention.

FIG. 4 is a diagram illustrating the ultraviolet light irradiation system according to the present invention.

FIG. 5 is a diagram illustrating the ultraviolet light irradiation system according to the present invention.

FIG. 6 is a diagram illustrating the ultraviolet light irradiation system according to the present invention.

FIG. 7 is a diagram illustrating the ultraviolet light irradiation system according to the present invention.

FIG. 8 is a diagram illustrating an optical waveguide of the ultraviolet light irradiation system according to the present invention.

FIG. 9 is a diagram illustrating the optical waveguide of the ultraviolet light irradiation system according to the present invention.

FIG. 10 is a diagram illustrating the optical waveguide of the ultraviolet light irradiation system according to the present invention.

FIG. 11 is a diagram illustrating the optical waveguide of the ultraviolet light irradiation system according to the present invention.

### DESCRIPTION OF EMBODIMENTS

Embodiments of the present invention will be described with reference to the accompanying drawings. The embodiments described below are examples of the present invention, and the present invention is not limited to the following embodiments. Note that, in the present specification and the drawings, the components having the same reference numerals indicate the same components.

### Embodiment 1

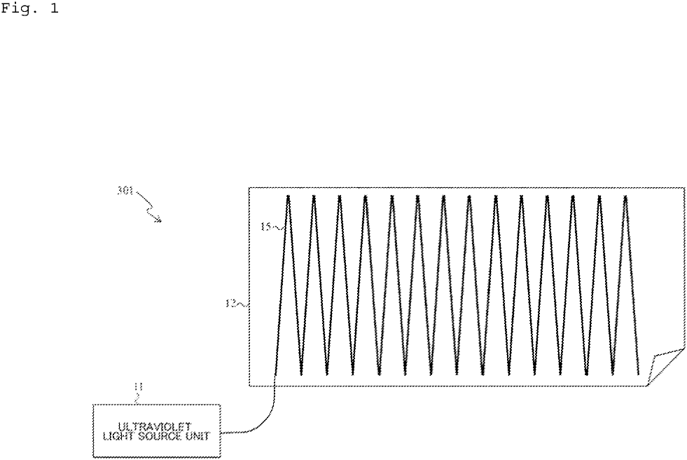

FIGS. 1 to 3 are diagrams illustrating an ultraviolet light irradiation system 301 of the present embodiment. The ultraviolet light irradiation system 301 includes an ultraviolet light source unit 11 for outputting ultraviolet light and a sheet 12 on which an optical waveguide 15 that laterally emits the ultraviolet light source unit 11 in a longitudinal direction is disposed.

The ultraviolet light source unit 11 emits light waves in a deep ultraviolet wavelength region having a wavelength of 200 to 300 nm. Specifically, light waves with a wavelength of 222 nm are preferable because they are known to have a sufficiently small influence on the human body. The ultraviolet light source unit 11 may include a light source having a wavelength longer than that of ultraviolet light and a harmonic generator. For example, the ultraviolet light source unit 11 may include a high-output light source of a 1064 nm band and a fourth or fifth harmonic generator.

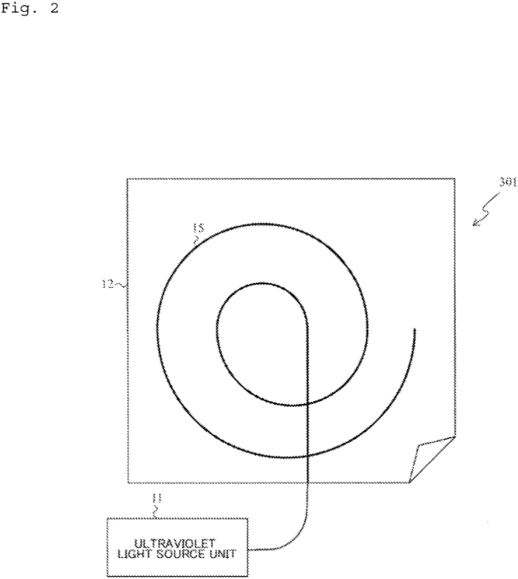

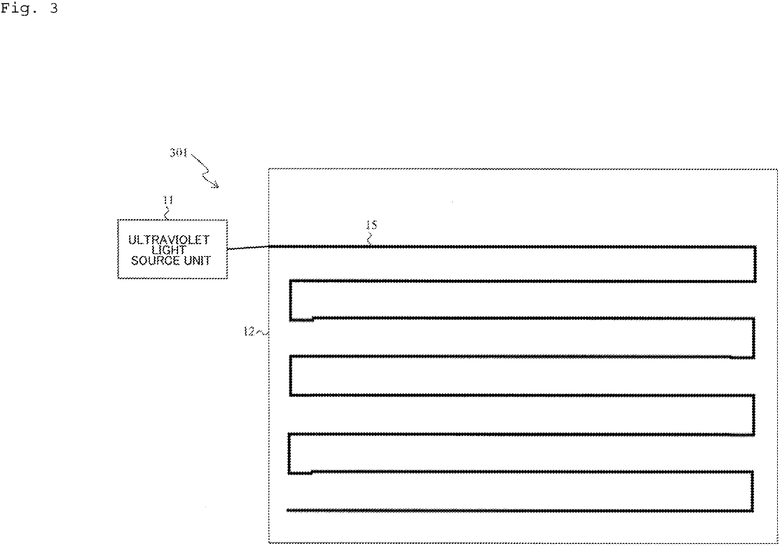

The optical waveguide 15 is disposed on the sheet 12. The optical waveguides 15 may be disposed in a zigzag shape as illustrated in FIGS. 1 or 3, or may be disposed in a spiral shape as illustrated in FIG. 2. The optical waveguide 15 is, for example, an optical fiber. The optical fiber transmits the ultraviolet light input from the ultraviolet light source unit 11 to the near end toward the far end while radiating the ultraviolet light from the optical fiber side surface.

Examples of the lateral radiation method of the optical fiber include a method of adding a material having a high scattering coefficient to a core, a method of forming a grating in the optical fiber, a method of giving minute bending to the optical fiber with minute ruggedness, a method of giving arbitrary bending (for example, a zigzag shape or a spiral shape) to the optical fiber, and the like. These methods will be described later.

As illustrated in FIGS. 1 or 2, the sheet 12 can be exemplified by a form in which an optical fiber is attached to a sheet formed from an arbitrary material, a form in which an optical fiber is built in a sheet formed from an arbitrary material, or a form such as a cloth or paper formed by weaving the optical fiber

As a method of using the ultraviolet light irradiation system 301 illustrated in FIGS. 1 and 2, such a sheet 12 is attached to a decontamination place, and the ultraviolet light is incident from the ultraviolet light source unit 11. The decontamination place can be decontaminated steadily by the ultraviolet light leaking from the optical waveguide 15. Alternatively, an article is manufactured from a cloth or paper formed by weaving optical fibers, and the article itself is decontaminated by making ultraviolet light incident from the ultraviolet light source unit 11.

Also, as illustrated in FIG. 3, in another example of the form of the sheet 12, the optical waveguide 15 for lateral radiation is formed in a glass film or a plastic film. The sheet 12 illustrated in FIG. 3 can be produced by a manufacturing technique of a planar lightwave circuit (PLC). In addition, in the sheet 12 illustrated in FIG. 3, the optical waveguide 15 may be formed inside a glass plate or a plastic plate by a laser processing technique. For example, it is preferable to use glass having excellent ultraviolet transmission characteristics and a high OH group concentration as the glass plate. In addition, it is preferable to use a glass material having a high scattering coefficient or to form an optical waveguide having a low NA with weak light confinement in order to laterally radiate the light from the optical waveguide 15.

As a method of using the ultraviolet light irradiation system 301 illustrated in FIG. 3, for example, the sheet 12 is attached to an object operated by an unspecified number of people (such as an operation panel of an ATM), and the ultraviolet light is incident from the ultraviolet light source unit 11. Even if a user touches the sheet 12, the sheet can be decontaminated steadily with the ultraviolet light leaking from the optical waveguide 15.

### Embodiment 2

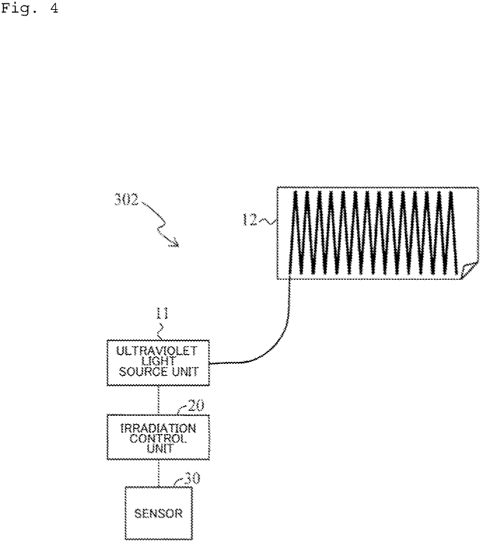

FIG. 4 is a diagram illustrating an ultraviolet light irradiation system 302 according to the present embodiment. For the ultraviolet light irradiation system 301 of Embodiment 1, the ultraviolet light irradiation system 302 further includes

a sensor 30 for detecting approach of a human body, and an irradiation control unit 20 configured to control output or non-output of the ultraviolet light from the ultraviolet light source unit 12 based on a signal of the sensor 30.

An UV-C region having a wavelength of 100 to 280 nm has a high decontamination effect, but is feared to have an influence on the human body. In a case where the UV-C region is used as ultraviolet light, it is preferable that the presence of a person or an animal is detected by the sensor 30 and the sensor signal is detected by the irradiation control unit 20 to control the operation of the ultraviolet light source 11. Further, even in a case where the ultraviolet light in the UV-C region is not used, it is possible for the irradiation control unit 20 to irradiate or not irradiate the ultraviolet light at an arbitrary timing, and it is preferable to improve safety and prolong the service life of the ultraviolet light source unit 11.

### Embodiment 3

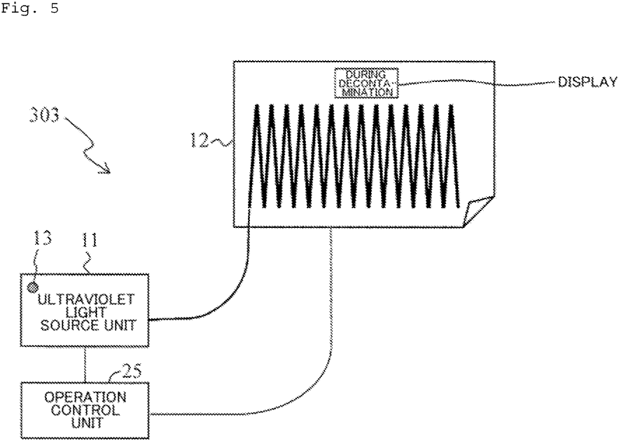

FIG. 5 is a diagram illustrating an ultraviolet light irradiation system 303 according to the present embodiment. For the ultraviolet light irradiation system 301 of Embodiment 1, the ultraviolet light irradiation system 303 further includes means for displaying that the ultraviolet light source 11 is outputting the ultraviolet light.

When an operation control unit 25 detects that the ultraviolet light source 11 is outputting the ultraviolet light, it notifies the user of the fact as follows.

### Notification by Vibration

The sheet 12 has vibration means, and when it is detected that the ultraviolet light source 11 is outputting the ultraviolet light, the operation control unit 25 vibrates the vibration means, and notifies the user that the ultraviolet light is being output.

### Display

The sheet 12 has display means, and when it is detected that the ultraviolet light source 11 is outputting the ultraviolet light, the operation control unit 25 causes the display means to display that decontamination is being performed and notifies the user that the ultraviolet light is being output. In addition, the ultraviolet light source unit 11 or the like is provided with a lamp 13, and when the ultraviolet light is being output, the lamp 13 is turned on to notify the user that the ultraviolet light is being output.

### Embodiment 4

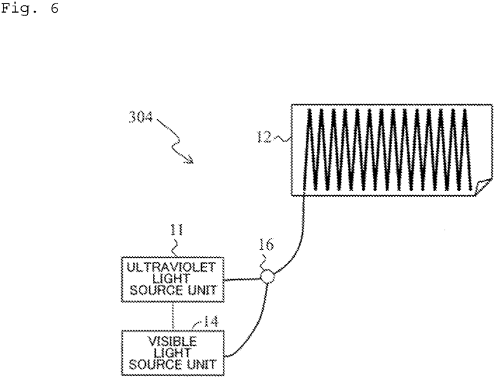

FIG. 6 is a diagram illustrating an ultraviolet light irradiation system 304 according to the present embodiment. For the ultraviolet light irradiation system 301 of Embodiment 1, the ultraviolet light irradiation system 304 further includes

a visible light source 24 that outputs visible light in synchronization with the output or the non-output of the ultraviolet light to the ultraviolet light source unit 11, and an optical multiplexing unit 16 that multiplexes the visible light output from the visible light source 14 with the ultraviolet light output from the ultraviolet light source unit 11.

The visible light is also laterally radiated from the optical waveguide 15. Therefore, the user can view the visible light leaking from the optical waveguide 15 during decontamination with the ultraviolet light, and can grasp that decontamination is being performed.

### Embodiment 5

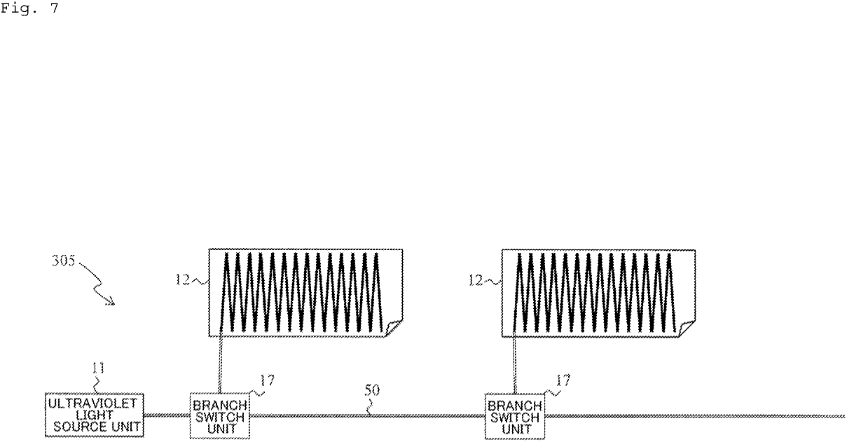

FIG. 7 is a diagram illustrating an ultraviolet light irradiation system 305 according to the present embodiment. For the ultraviolet light irradiation system 301 of Embodiment 1, the ultraviolet light irradiation system 305 further includes a plurality of sheets 12, and

a branch switch unit 17 configured to branch the ultraviolet light output from the ultraviolet light source unit 11 and supply the branched ultraviolet light to the optical waveguide 15 of each sheet 12, or sequentially supply the ultraviolet light output from the ultraviolet light source unit 11 to the optical waveguide 15 of each sheet 12.

The ultraviolet light irradiation system 305 transmits the ultraviolet light emitted from one ultraviolet light source 11 through an optical fiber 50, branches the ultraviolet light by the branch switch unit 17, and supplies the branched ultraviolet light to a plurality of sheets 12. Alternatively, the ultraviolet light irradiation system 305 transmits the ultraviolet light emitted from one ultraviolet light source 11 through the optical fiber 50, switches the route at arbitrary timing or at constant intervals by the branch switch unit 17, and sequentially supplies the ultraviolet light to each sheet 12. In this case, the object to be decontaminated is changed at arbitrary timing or at fixed intervals.

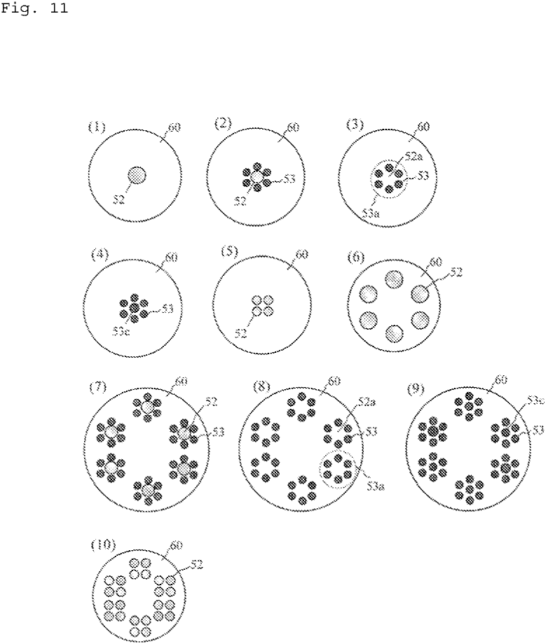

As the optical fiber 50 for supplying ultraviolet light to the sheet 12, an optical fiber having a cross section as illustrated in FIG. 11 can be used. In addition to the solid type optical fiber using a general additive such as that illustrated in (1) of FIG. 11, optical fibers having a hole structure described in (2) to (4) of FIG. 11, multi-core optical fibers having a plurality of core regions described in (5) and (6) of FIG. 11, or optical fibers having a structure obtained by combining them ((7) to (10) of FIG. 11) may be used.

FIG. 11 is a diagram illustrating a cross section of the optical fiber. The optical fiber having a cross-sectional structure as illustrated in FIG. 11 can be used as the optical fiber 50.

### Solid Core Optical Fiber

The optical fiber has one solid core 52 with a refractive index higher than that of a clad 60 in the clad 60. The “solid” means “not a cavity”. The solid core can also be realized by forming an annular low refractive index region in the clad.

### Hole Assist Optical Fiber

The optical fiber has the solid core 52 and a plurality of holes 53 disposed on the outer periphery of the solid core 52 in the clad 60. The medium of the hole 53 is air, and the refractive index of the air is sufficiently smaller than that of quartz glass. Therefore, the hole assist optical fiber has a function of returning light leaked from the core 52 by bending or the like to the core 52 again, and has a characteristic of small bending loss.

### Hole Structure Optical Fiber

The optical fiber has a hole group 53a of a plurality of the holes 53 in the clad 60, and has a refractive index effectively lower than that of a host material (glass or the like). This structure is called a photonic crystal fiber. This structure can have a structure in which a high refractive index core having a varied refractive index does not exist, and light can be confined by making a region 52a surrounded by the holes 53 as an effective core region. As compared with an optical fiber having a solid core, a photonic crystal fiber can reduce the influence of absorption and scattering loss of the core by an additive, and can realize optical characteristics which cannot be realized by the solid optical fiber such as reduction of bending loss and control of nonlinear effect.

### Hollow Core Optical Fiber

The core region of the optical fiber is formed with air. The light can be confined in the core region by taking a photonic band gap structure by a plurality of holes or an anti-resonant structure by a glass thin wire in the clad region. This optical fiber has a small nonlinear effect and can supply a high output or high energy laser.

### Coupled Core Type Optical Fiber

In this optical fiber, a plurality of the solid cores 52 with a high refractive index are disposed in close proximity to each other in the clad 60. The optical fiber guides light by light wave coupling between the solid cores 52. Since the coupled core type optical fiber can disperse and send light by the number of cores, it is possible to increase the power and sterilize efficiently as much. Furthermore, the coupled core type optical fiber has an advantage that the fiber deterioration due to ultraviolet rays can be alleviated and the service life can be prolonged.

### Solid Core Type Multi-Core Optical Fiber

In this optical fiber, a plurality of solid cores 52 with a high refractive index are disposed in the clad 60 apart from each other. This optical fiber guides light in a state where the influence of the light wave coupling can be ignored by sufficiently reducing the light wave coupling between the solid cores 52. Therefore, the solid core type multi-core optical fiber has the advantage that each core can be handled as an independent waveguide.

### Hole Assist Type Multi-Core Optical Fiber

The optical fiber has a structure in which a plurality of hole structures and core regions of the above-mentioned (2) are disposed in the clad 60.

### Hole Structure Type Multi-Core Optical Fiber

The optical fiber has a structure in which a plurality of hole structures of the above-mentioned (3) are disposed in the clad 60.

### Hollow Core Type Multi-Core Optical Fiber

The optical fiber has a structure in which a plurality of hole structures of the above-mentioned (4) are disposed in the clad 60.

### Coupled Core Type Multi-Core Optical Fiber

The optical fiber has a structure in which a plurality of hole structures of the above-mentioned (5) are disposed in the clad 60.

Although a plurality of ultraviolet light irradiation systems 301 described in Embodiment 1 may be prepared and arranged for each decontamination object, the number of ultraviolet light sources 11 can be reduced because ultraviolet light emitted from one ultraviolet light source 11 is shared by a plurality of sheets 12 in the ultraviolet light irradiation system 305. Further, by combining a plurality of sheets, the decontamination range can be expanded, and reliability can be improved, such as the operation of the other part even if a part of the sheet is defective.

### Example of Optical Waveguide

### Laterally Radiate Light With External Force

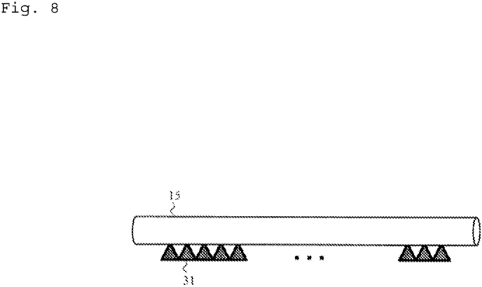

In a case where the optical waveguide 15 is an optical fiber, the lateral radiation of ultraviolet light in the optical fiber can be realized by applying external force to the optical fiber for ultraviolet light transmission at an arbitrary point. For example, radiation by bending as illustrated in FIGS. 1 and 2, and radiation (micro bend loss) by a minute ruggedness imparting unit 31 as illustrated in FIG. 8 can be exemplified.

### Laterally Radiate Light by Material, Manufacturing Method, and Processing

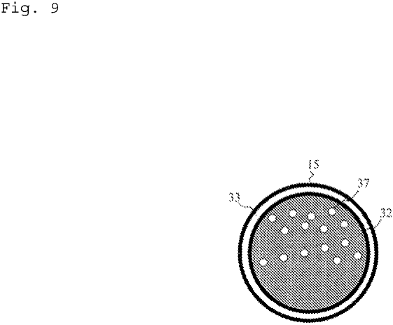

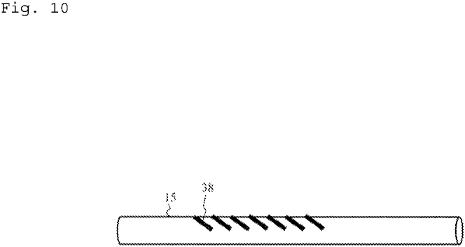

In a case where the optical waveguide 15 is an optical fiber, the lateral radiation can be realized by the material, manufacturing method, and processing of the optical fiber. For example, there are methods of using a glass material with a high scattering coefficient for the core, intentionally generating a bubble (scatterer 37) in a core region 32 in a preform or spinning step as illustrated in FIG. 9, or applying a scratch 38 (grating) to the inside of an optical fiber by laser processing as illustrated in FIG. 10. Reference numeral 33 denotes a cladding region.

As the optical fiber of the optical waveguide 15, an optical fiber having a cross section as illustrated in FIG. 11 can be used. In addition to the solid type optical fiber using a general additive such as that illustrated in (1) of FIG. 11, optical fibers having a hole structure described in (2) to (4) of FIG. 11, multi-core optical fibers having a plurality of core regions described in (5) and (6) of FIG. 11, or optical fibers having a structure obtained by combining them ((7) to (10) of FIG. 11) may be used. Particularly, in a case where the multi-core optical fiber is used, it is preferable that the radiation direction is controlled by the core arrangement and that the input and output are increased by the dispersion of the transmission light.

In a case where the lateral radiation is realized by the method of the above-mentioned (2), the scatterer 37 in the core region 32 is disposed to be deviated in a certain direction or the position of the scratch 38 by processing is arranged to be deviated in a part in the circumferential direction, so that strong radiation can be obtained in the direction where the scatterer 37 or the scratch 38 exists from the center of the core. Thus, the decontamination effect in a specific surface of the sheet 12 or the like is improved, which is preferable. The scatterer 37 of the core may be arbitrarily disposed, for example, by drawing up rods of high scattering glass and ordinary glass to produce a preform, or by generating bubbles at predetermined positions in the fiber by laser processing or the like.

### EXAMPLES

A specific example is described below.

In the ultraviolet light irradiation system of the present embodiment, decontamination is performed by attaching the sheet 12 on which the optical waveguide 15 that laterally emits light in a longitudinal direction is disposed, and inputting ultraviolet light to the optical waveguide 15 of the sheet 12.

The first specific example is an example in which the optical waveguide 15 is woven into a cloth (sheet 12) of a seat of a train or the like. The ultraviolet light source 11 is disposed, for example, other than an area used by a person (for example, below a seat), propagates ultraviolet light through the optical fiber 15 and supplies the ultraviolet light to the optical waveguide 15 woven into the sheet 12. The seat can be decontaminated with the ultraviolet light before or after train operation in a time when no passenger is present. Similarly, an optical waveguide 15 is woven, wound or attached to a part touched by an unspecified number of people such as a strap/handrail part of a train, a handrail part of an escalator, and the like to decontaminate the parts, and thus, the user can use the parts without any input from a user of the decontamination.

In addition, if the light is the ultraviolet light having a wavelength (for example, a wavelength of 222 nm) that have little effect on the human body, the light can be always supplied from the ultraviolet light source 11, and the decontamination of the seat can be always performed.

The second specific example is an example in which the sheet 12 is disposed on the surface of a touch panel provided in an ATM, an automatic ticket vending machine, or the like. As described in Embodiment 2, in this example, the ultraviolet light source 11 supplies the ultraviolet light in the UV-C region (for example, a wavelength of 254 nm) to the optical waveguide 15 of the sheet 12. As described in Embodiment 2, the ultraviolet light source 11 supplies ultraviolet light only when a person does not operate the touch panel by using the sensor 30, and decontaminates the touch panel. Similarly, the decontamination of the buttons or the like can be completed without any input from a user of the user by attaching the sheet 12 to a part to be used by the unspecified number of people such as buttons of an elevator and a vending machine.

Reference Signs List

| | |
|---|---|
| 11 | Ultraviolet light source |
| 12 | Sheet |
| 13 | Lamp |
| 14 | Visible light source |
| 15 | Optical waveguide |
| 16 | Optical multiplexing unit |
| 17 | Branch switch unit |
| 20 | Irradiation control unit |
| 25 | Operation control unit |
| 30 | Sensor |
| 31 | Ruggedness imparting unit |
| 32 | Core region |
| 33 | Cladding region |
| 37 | Scatterer |
| 38 | Scratch |
| 50 | Optical fiber |
| 52 | Solid core |
| 52 a | Region |
| 53 | Hole |
| 53 a | Hole group |
| 60 | Clad |
| 301 to 305 | Ultraviolet light irradiation system |

1. An ultraviolet light irradiation system comprising:
an ultraviolet light source unit for outputting ultraviolet light; and
a sheet on which an optical waveguide that laterally emits the ultraviolet light in a longitudinal direction is disposed.

2. The ultraviolet light irradiation system according to claim 1, further comprising:
a plurality of the sheets; and
a branch switch unit configured to branch the ultraviolet light output from the ultraviolet light source unit and supply the branched ultraviolet light to the optical waveguide of each sheet, or sequentially supply the ultraviolet light output from the ultraviolet light source unit to the optical waveguide of each sheet.

3. The ultraviolet light irradiation system according to claim 1, further comprising:
a sensor for detecting approach of a human body; and
an irradiation control unit configured to control output or non-output of the ultraviolet light from the ultraviolet light source unit based on a signal of the sensor.

4. The ultraviolet light irradiation system according to claim 1, further comprising:
a visible light source that outputs visible light in synchronization with output or non-output of the ultraviolet light of the ultraviolet light source unit; and
an optical multiplexing unit that multiplexes the visible light output from the visible light source with the ultraviolet light output from the ultraviolet light source unit.

5. The ultraviolet light irradiation system according to claim 1, wherein
the optical waveguide is provided with predetermined bending or micro-bending.

6. The ultraviolet light irradiation system according to claim 1, wherein
the optical waveguide has a plurality of bubbles, refractive index inhomogeneous points, or gratings in a waveguide region of the ultraviolet light.

7. The ultraviolet light irradiation system according to claim 1, wherein
the optical waveguide includes any one of a solid core optical fiber, a hole assist optical fiber, a hole structure optical fiber, a hollow core optical fiber, a coupled core type optical fiber, a solid core type multi-core optical fiber, a hole assist type multi-core optical fiber, a hole structure type multi-core optical fiber, a hollow core type multi-core optical fiber, and a coupled core type multi-core optical fiber.

8. A decontamination method comprising:
attaching a sheet on which an optical waveguide that laterally emits light in a longitudinal direction is disposed to a desired object; and
inputting ultraviolet light into the optical waveguide of the sheet.
